(19) United States
(12) Patent Application Publication
(10) Pub. No.: US 2012/0283615 A1
(43) Pub. Date: Nov. 8, 2012

(54) PRESSURE SENSITIVE ADHESIVE FOR APPLICATION ON SKIN AND PROCESS FOR THE PRODUCTION THEREOF

(75) Inventors: Ranjit MALIK, York, PA (US); Kevin MCKINNEY, Glen Rock, PA (US)

(73) Assignee: ADHESIVES RESEARCH, INC., Glen Rock, PA (US)

(21) Appl. No.: 13/459,679

(22) Filed: Apr. 30, 2012

Related U.S. Application Data
(60) Provisional application No. 61/481,948, filed on May 3, 2011.

(51) Int. Cl. A61F 13/02

(52) U.S. Cl. 602/52; 602/54; 523/111

(57) ABSTRACT

Pressure sensitive adhesives for use primarily in medical applications are disclosed. The pressure sensitive adhesives are urethane elastomeric adhesives that include a polyurethane polymer and a compatible tackifier and/or compatible plasticizer to achieve an adhesive having a predetermined compliance. The result is a pressure sensitive adhesive for application to skin that can eventually be removed with very little trauma to the skin and with a low pain rating, but which still exhibits very good peel strength when used to construct an article in which the adhesive is adhered to human skin.

### RELATED APPLICATION

This application claims the benefit of and priority to U.S. Provisional Application No. 61/481,948, filed May 3, 2011, which is hereby incorporated by reference in its entirety.

### FIELD

The present application is directed toward the field of pressure sensitive adhesive compositions and their production and more particularly to such compositions adapted for medical applications such as wound care and the like.

### BACKGROUND

Most pressure sensitive adhesives currently used in connection with dressings for skin and wound-care bond tenaciously to the skin. The level of bond strength builds up even after just a few hours of wear. The sensory perception felt when peeling back such adhesives that have had even just a few hours to dwell on the skin is typically quite painful and causes damage to the epidermal layer of the skin. Pain can be caused by trauma to the skin by way of induced edema and/or erythema.

Furthermore, dressings are often used to repeatedly cover the same site of the body, resulting in repeated removal and reapplication of the adhesive. When repeatedly applied and removed, such adhesives are apt to remove with them parts of the upper skin and cause damage to the skin. The damage to the skin can manifest in an increase in transdermal water loss. These adhesives also fasten strongly to hair on the skin, which adds to the discomfort and irritation experienced when the adhesive is removed. Additionally, the skin layer stripped by the adhesive during removal deadens the tack and the adhesive properties, thus diminishing the reapplication potential of the adhesive.

Gel adhesives provide an alternative to pressure-sensitive adhesives and are more gentle to the skin. A gel adhesive has a low peel with skin and can be removed with little damage and it typically wets out the surface well. However, gel adhesives have shown poor adhesive performance because the tape or dressing easily gets caught on clothing or other surfaces and rolls off. The adhesive bond does not have the strength to overcome the rolling action. Once the edge of the adhesive rolls, it readily picks up lint and other debris that will prevent it from re-bonding to the skin and eventually the product will fail. Additionally, the edge roll can lead to contamination of the healing site. As a result, gel adhesives do not have sufficient wear properties for most wound care applications. Common gel adhesives include silicone and polyurethane gels, although most of the polymers for polyurethane gel adhesives are made using catalysts that render the adhesive cytotoxic and, as a result, are not suitable for application on skin.

Silicone gel adhesives are sometimes used in wound applications, since most do not have cytotoxicity concerns. However, these adhesives still do not bond well to the skin if the adhesive thickness is less than 80 microns, and typically up to 150 microns. Yet while the initial bond to the skin improves with increasing thickness, the overall wear properties are not improved due to edge lift resulting from the larger exposed adhesive area at the edge of the dressing. This exposed adhesive edge tends to “grab” clothing, bedding or other contacted materials which then results in the edge lifting. In the worst case, edge lift results in rolling of the dressing to the point of complete removal. Yet another drawback is that the cost of silicone gels is substantially higher than other commercially available adhesives. Furthermore, ionizing radiation, often used for sterilization of wound dressings, can have a particularly detrimental effect on silicone gel adhesives, resulting in much lower bonds to the skin. For this reason, silicone gel adhesives are usually sterilized using an ethylene oxide sterilization procedure, a more expensive process that further contributes to higher manufacturing costs.

In order to address the desire for pain free removal, the concept of adhesive inactivation has also been discussed in the literature. Acrylic, polyurethane or rubber based adhesives may be used in conjunction with the deactivation method. The deactivatable adhesive can form strong bonds until it is time for removal. Using a trigger mechanism, at the time of removal, the adhesive is made to lose its bond strength. Various trigger mechanisms, such as light source, use of saline solution, and the use of microcapsules filled with oils have been proposed. A common drawback with any of the inactivation methods is that it is irreversible. Once inactivated the adhesive permanently loses its adhesive properties. The capability to reapply the adhesive is lost. Further disadvantage of adhesives that deactivate with light source is that these cannot withstand gamma or e-beam sterilization methods. Deactivation with the microcapsule method relies upon rupturing of microcapsules with application of pressure to the adhesive followed by migration of oil in the adhesive. Oil migration is a slow process. Further, it is extremely difficult to standardize the pressure for individual patients and inadvertent inactivation of the adhesive cannot be prevented. This is further complicated because applying pressure to a wound is not desirable.

Hydrogels and hydrocolloids can also be formulated to provide a more gentle adhesive, however the adhesive properties of these materials change dramatically as they absorb or lose water from/to the environment or wound site, often resulting in adhesive/skin bond failure. Hydrogels and hydrocolloids are also applied as thick layers of greater than 80 microns due to their weak bonds to the skin at lower adhesive thicknesses.

Yet another drawback of silicone, rubber, and acrylic-based pressure sensitive adhesives currently used with wound care is that these adhesives generally have poor moisture vapor transmission rate (MVTR) performance. In order to improve the MVTR in wound-care dressings for these types of adhesives, one or more of the following measures is typically taken: the adhesive is coated as a very thin layer, generally less than 30 microns; the adhesive is pattern coated on the web to create a substantial adhesive-free area to allow for moisture transmission; the adhesive is coated to form a porous or microporous structure so that the skin can breathe; and/or the adhesive is perforated to create an adhesive-free area.

The use of a low coating thickness can be inconsistent with achieving a threshold adhesive mass for good bond formation, while pattern coating, formation of pores, and perforation may necessitate additional process steps in manufacture, which can increase manufacturing costs. Furthermore, in some cases, the presence of pores and holes may be undesirable as they may allow ingress of bacteria into the wound. In some cases, the adhesive free areas resulting from pattern coatings may compromise wear performance.

Accordingly, there is a need in the medical and wound care marketplace for an adhesive that can be removed from the skin with little or no pain and for such an adhesive that also exhibits suitable wear performance. There is a further need for adhesives that have the ability to be repositioned on the skin if the adhesive were misapplied in the initial application as well as reapplied after removal for wound inspection. There is still a further need to provide such adhesives that can be manufactured and sold in a cost effective manner. There is also a need for an adhesive that retains performance characteristics upon gamma sterilization. There is also a need for an adhesive that has high MVTR characteristics.

### SUMMARY

Exemplary embodiments of the present invention provide a pressure sensitive adhesive that address these and other deficiencies currently found in the art. The PSA can be used in fabricating medical devices such as affixing tapes, wound dressings, and other articles in contact with human skin.

According to one embodiment, a medical pressure sensitive adhesive is provided that comprises a polyurethane elastomer and a plasticizer or tackifier, wherein the adhesive has compliance properties corresponding to a TA Peak Force in the range of 40 grams to 180 grams and a percent loss in the range of 40% to 95%.

According to another embodiment, an article comprises a substrate and a medical pressure sensitive adhesive laminated to the substrate, the adhesive comprising a polyurethane elastomer and a plasticizer or tackifier, wherein the adhesive has compliance properties corresponding to a TA Peak Force in the range of 40 grams to 180 grams and a percent loss in the range of 40% to 95%.

In certain embodiments, the article has a stripping effect of a maximum of 20% when the adhesive is applied to human skin. In certain embodiments, the average pain rating upon removal of the adhesive from human skin is less than 2.5 on the Wong Baker pain scale. In certain embodiments, the adhesive exhibits a moisture vapor transmission rate value of greater than 1000 g/m²-day. In certain embodiments, the article exhibits edge lift of less than 5% after 24 hours when applied to human skin. In certain embodiments, the adhesive has a peel strength of greater than 0.8 N/cm after 24 when applied to human skin. In preferred embodiments, all of these properties are exhibited.

An advantage of exemplary embodiments is that a pressure sensitive adhesive is provided for use in medical applications that has sufficient adhesive strength to hold a dressing or other article in place but that can be removed with very little trauma to the skin or corresponding pain to the patient.

Another advantage is that the adhesive permits a dressing or other medical article to which it is applied to be removed and subsequently re-attached without relevant loss in adhesive strength.

Still another advantage is that the adhesive does not exhibit a loss of tack such that if a bandage or other medical article is misapplied or folds over during application, it can be reworked and applied correctly without the need to obtain a new bandage.

Other features and advantages of the present invention will be apparent from the following more detailed description of exemplary embodiments, which illustrate, by way of example, the principles of the invention.

### BRIEF DESCRIPTION OF THE DRAWINGS

FIGS. 1 and 2 are a side and top view, respectively, of a medical article employing an adhesive in accordance with an exemplary embodiment.

### DETAILED DESCRIPTION OF EXEMPLARY EMBODIMENTS

Pressure sensitive adhesives in accordance with exemplary embodiments are urethane elastomeric adhesives that comprise a polyurethane polymer and a compatible tackifier and/or compatible plasticizer to achieve an adhesive having a predetermined compliance. In some embodiments, the adhesive is then coated on a substrate, followed by subsequent drying/curing by passing it through a hot air oven or alternatively under UV light. The resulting product is a pressure sensitive adhesive for application to skin that can eventually be removed with very little trauma to the skin and with a low pain rating.

Exemplary embodiments are thus directed to a urethane elastomeric pressure sensitive adhesive that can be removed from the skin with little to no pain and with little or no trauma to skin but which also can easily be reapplied or repositioned and resists edge rolling when used in conjunction with a tape, dressing, or other article. The polyurethane pressure sensitive adhesives in accordance with exemplary embodiments provide the ability to lift-up a dressing for inspection of a wound and then to re-attach the adhesive without relevant loss in adhesive strength, which is highly advantageous in a clinical setting. This also allows a clinician to rework the adhesive patch in case it is misapplied or folds over on itself. This simplifies handling and also saves time for clinical staff.

Accordingly, exemplary embodiments are further directed to the use of urethane elastomeric pressure sensitive adhesives in connection with medical applications such as wound care, incise drapes, securing intravenous sites, and vacuum assisted closure and the present invention also relates to a wound dressing, affixing tape or other medical device for skin application that includes a substrate coated on at least one side with the adhesives described herein. In addition to wound dressings and affixing tapes, the urethane elastomeric pressure sensitive adhesives can be used in fabricating medical devices such as medical drapes, IV site dressings, surgical gowns and drapes, transdermal delivery systems, medical device fixation, and other medical devices for attaching to the skin, all by way of example only.

Exemplary embodiments provide adhesives in which the pain experienced on removal is low, even after up to 5 days of wear; the adhesion does not significantly build with time; the adhesive has a MVTR in excess of 1000 g/m² per day; does not cause maceration of the skin; and the surface of the adhesive is substantially free of skin cells when the adhesive is peeled back.

The base polymer for pressure sensitive adhesives in accordance with exemplary embodiments is a polyurethane. The polyurethane may be any suitable biocompatible, hydrophilic polyurethane which can be modified such that the formulated urethane elastomer pressure sensitive adhesive exhibits a compliance having a TA Peak Force in the range of 40 grams to about 180 grams and a percent loss in the range of 40% to 95%, as described in more detail herein. Conventional PSAs used as medical adhesives have a TA Peak Force in the range of 200 to 500 grams. The inventors unexpectedly discovered that PSAs that exhibit a TA Peak Force of 180 grams or less result in the pain sensation on skin being greatly diminished. The compliant urethane elastomer adhesives used in accordance with exemplary embodiments have weaker adhesive bonds to the skin than the pressure sensitive adhesives normally used for adhesive dressings. Consequently, these elastomers leave the stratum corneum essentially intact when dressings containing these elastomers are peeled or pulled away from the skin.

In one embodiment, the polyurethane is comprised of a plurality of urethane linkages and is substantially free of isocyanate functionality (i.e., in which no NCO content is detected by FTIR spectroscopy). The polyurethane typically has a weight average molecular weight in excess of 25,000 g/mol, preferably between 50,000 and 150,000 g/mol. The polyurethane is further selected to be soft and elastomeric, but with high strength. The glass transition temperature of the polyurethane is preferably less than −25° C. and the compliance can be readily modified. Furthermore, the polyurethane should not absorb water but be capable of transporting large amounts of moisture.

The polyurethane may be formed through polymerization of commercially available polyurethane resins with terminal acrylic functionality or mixtures thereof, including those available from Bomar Specialties as BR-3641AA, BR-3731, and BR-3741AB or any one of CN3211, CN9004, CN9018, CN9290US, or CN9782, available from Sartomer, by way of example only. Alternatively, the polyurethane may be obtained as a pre-polymerized PSA solution. Exemplary such polymers include the polyurethane-based pressure-sensitive adhesive available as Cyabine SP-205 produced by Toyo Ink Mfg. Co., Ltd., again by way of example only. In still other embodiments, the polyurethane can be obtained by the reaction of an isocyanate terminated resin with a hydroxyl terminated resin. Exemplary materials include reacting isocyanate resins such as Mondur and Desmodur available from Bayer with polyols such as Acclaim, Arcol, Desmophen, or Multranol, all of which are also available from Bayer.

Exemplary adhesive formulations further include a tackifier and/or plasticizer. The plasticizer is selected for its biocompatibility and its ability to modify the compliance of the adhesive formulation and to achieve the other properties described herein. The plasticizer should be non-volatile and be insoluble in water and should also not absorb water or other bodily fluids. The adhesive formulation is typically about 10% by weight to about 60% by weight plasticizer, and in some cases may be in the range of about 20% by weight to about 55% by weight plasticizer. In some embodiments, the plasticizer may be present in the range of about 30% to about 40% by weight.

The plasticizer is selected to be compatible with polyurethane across a range of loading levels, forming a single phase and exhibiting little or no sweating out with increasing time and/or temperature. Furthermore, it should be selected such that the resulting formulated adhesive does not leave residue on the skin upon removal. It will thus be appreciated that the selection of a particular plasticizer may depend upon the particular polyurethane elastomer used as the base component of the PSA.

Suitable plasticizers may include triisodecyl trimellitate; tributyl trimellitate; tri-n-hexyl trimellitate; tris n-(C7-11)alkyl ester branched and linear 1,2,4 benzenetricarboxylic acid; butyl benzoate; di-ethylhexylphthalate; di-octylphthalate; di-butylphthalate; diethylhexyl adipate; dibutyl adipate; triethyl citrate; tributyl citrate; acetyl triethyl citrate; acetyl trin-butyl citrate; n-butyryl tri-n-hexyl citrate; triacetin; glycerin; caprylic/capric triglyceride; tricaprin; tricaprylin; propylene glycol dicaprate; propylene glycol dicaprylate/dicaprate; poly(ethylene glycol) (PEG); hydrogenated vegetable oil; hydrogenated seed oil; PEG dilaurate; PEG diethylhexylonate; and combinations thereof.

Exemplary adhesive formulations further may include up to about 50% by weight of a tackifier. The tackifier may be selected from the group consisting of rosin esters, polymerized rosins, hydrogenated rosins, polyterpenes, styrenated terpenes, polymerized hydrocarbon resins, alpha methyl styrenes, alpha methyl styrene phenolics and combinations thereof. Exemplary tackifiers include those which are commercially available as Escorez 1310, Sylvares SA120, Sylvares TP105, Foral 85, and Sylvares 540. As with the plasticizer, the tackifier is selected for its biocompatibility (i.e., its ability to be safely in contact with the skin and/or bodily fluids) and compatibility with (i.e., its ability to form a single phase with) the polyurethane resin.

Despite the presence of plasticizers and tackifiers, which are relatively small molecules compared to the base polymer, exemplary embodiments exhibit little to no migration of the plasticizer or tackifiers from the adhesive because of their compatibility with the polyurethane polymer, having closely matched solubility parameters and being completely miscible in one another over an expected temperature range of adhesive storage and use.

Exemplary embodiments may also employ a cross-linking agent. The cross-linking agent may be present up to about 5% by weight of the polyurethane on a solids basis, more typically up to about 3% by weight of the polyurethane on a solids basis, and in some cases in the range of about 0.75% to about 2.25% by weight of the polyurethane on a solids basis. Any suitable cross-linking agent may be used, including isocyanates and aziridines, and metal chelates such as Tyzor TBT, and Tyzor GBA (both available from Dupont), by way of example only.

Other additives in the types and amounts as are known in the art for use in conventional pressure-sensitive adhesives may also be employed, provided those additives do not adversely affect the properties otherwise sought to be achieved. Such additives may include antioxidants, stabilizing agents for enhanced shelf-life, and the like. Agents added to stabilize the adhesive against the detrimental effects of gamma sterilization include, but are not limited to, those commercially available as Irganox 1010, Irganox 1076, Irganox 245, Irganox 3052F, Irganox E201, Irganox B225, Ubiquinone, Tinuvin 662, and Tinuvin 770.

Pressure sensitive adhesives in accordance with exemplary embodiments used to affix materials to the body are compatible with skin. Biocompatibility of pressure sensitive adhesives is characterized by cytotoxicity, skin irritation, and skin sensitization. The cytotoxicity of adhesives in accordance with exemplary embodiments does not exceed 2 when using the ISO agrose overlay method; preferably the cytotoxicity is less than 1 and most preferably is zero. The skin irritation, using the ISO skin irritation rating, does not exceed 2 and preferably is less than or equal to 0.4 (non-irritating). Adhesives in accordance with exemplary embodiments do not act as skin sensitizers under Globally Harmonized System for Classification and Labeling of Chemicals (GHS) standards.

Exemplary embodiments result in adhesive compositions that can be applied to skin, such as in conjunction with the application of a dressing, affixing tape, or other medical device adhered to the skin and that can be subsequently removed with little or no pain. Although pain experienced during adhesive removal can be difficult to measure precisely as it can be influenced by a wide range of factors, the Wong-Baker pain scale is recognized in the medical field to quantify pain intensity measurement. This 0 to 5 scale, with 5 being the highest pain level, is often used to gauge the pain experience of an individual. Exemplary embodiments achieve an average Wong-Baker pain rating of less than 2.5 during adhesive removal even after up to 5 days of wear, typically less than 2.0, preferably less than 1.5, and in some cases less than 1.0.

The inventors have found that polyurethane based pressure sensitive adhesives falling within a specified compliance range are much gentler on the skin, while meeting the desired wear performance. Surprisingly, the inventors have further determined that the peel strength of the adhesive does not correlate with pain. Observation of the bond failure mechanism of conventional adhesives suggest that the bond failure when peeling the adhesive from skin does not take place at the adhesive-skin interface but instead the failure takes place at the interface between the upper layer of skin cells and the dermis. This is signified by the large quantity of skin cells fouling the peeled-back adhesive. Therefore, the force required to remove the adhesive from the skin is essentially the same as the force at which the adhesive pulls off large amounts of skin cells from the dermis layer (i.e., resulting in trauma to the skin and thus translating to pain felt by the wearer).

In the inventive adhesive the bond failure occurs at the adhesive-skin interface which is signified by none or very little skin cell fouling the adhesive. Using this underlying difference in the mechanism of bond failure when peeling from skin, the inventors were able to develop a unique system that possesses both high peel and low pain upon removal. Put yet another way, while the terms low-peel, low-trauma and low pain have generally been used interchangeably in the art, the inventors have determined that this usage is incorrect. Thus, while two adhesives could exhibit similar peel force, they may be very different in the pain experienced by the individual. It will be appreciated that the amount of skin cells removed during peeling may be very different for different adhesives despite a similar peel value. The higher the amount of skin cells removed, the higher the pain experienced during the removal.

Adhesives in accordance with exemplary embodiments have a stripping effect of less than 50%, i.e. are capable of being removed from the skin with less than 50% of the adhesive area being fouled by detached skin cells and typically the stripping effect is less than about 20%. In preferred embodiments, the stripping effect is less than about 10%, such that up to 90% or more of the previous bonding force is available so that the adhesive can be repositioned and re-attached to the skin. Furthermore, the removal of fewer skin cells correlates to less pain experienced by the wearer.

While it has been determined that peel force does not correlate with pain, it does correlate with wear performance. Exemplary embodiments also result in an adhesive that has suitable wear performance. If the peel is reduced too much, then the adhesive deteriorates in wear properties, i.e., tends to roll off or fall off prematurely. Thus, the peel force is preferably as close to, but not over, the amount of force required to remove a majority of skin cells from the area of the skin in contact with the adhesive, although it will be appreciated that force can vary slightly from person to person, based on skin type, weather conditions and diet, for example.

In order for the adhesive dressing or affixing tape for skin applications to function effectively, the force with which the compliant urethane elastomer adhesive adheres to the skin should exceed the load to which it is subjected during normal use. The peel force is on the order of 0.2 N force per centimeter of width when peeling or stripping at an angle of 90° from the skin. Typically the force is more than 0.4 N/cm, which allows for the vast majority of samples to bond to the skin for several days. Preferably, the peel force is 0.6 N/cm using a 1 hour dwell and over 0.8 N/cm after a 24 hour dwell on the skin.

One method of quantifying wear performance is edge lift. Edge lift is a measure of the percentage of the total area of a patch to which adhesive has been applied that is no longer bonded to the skin during the wear-time. Exemplary embodiments achieve less than 5% edge lift occurring over a 24 hour period.

Adhesives in accordance with exemplary embodiments also exhibit high MVTR (greater than 1000 g/m² day), an advantage in medical applications which allows the skin to breathe. Adhesives that do not breathe tend to accumulate moisture at the skin-adhesive interface which in turn leads to maceration of the skin. Macerated skin becomes weak and it can easily tear and cause pain when the adhesive is removed. Accumulation of moisture also promotes bacterial growth on the skin. A 25 micron thick layer of adhesive in accordance with exemplary embodiments may exhibit an MVTR of over 1500 g/m² day, while even a 44 micron thick layer of the adhesive coated on 25 micron polyurethane film can exhibit an MVTR of 1400 g/m² day.

Dressings and other devices making use of adhesives in accordance with exemplary embodiments can also exhibit little or no sliding or creep from the application site. They remove cleanly, leaving little to no residue on skin or clothing, even if contacted by fluids (e.g., water, isopropanol, wound exudate, etc).

Pressure sensitive adhesives in accordance with exemplary embodiments are highly compliant while having high elongation properties at break. The urethane elastomer pressure sensitive adhesives exhibit a compliance having a peak force between the range of about 40 to 180 grams and a percent loss in the range of about 40 to 95%. Compliance is measured using a Texture Analyzer, such as a TA-XT2i Texture Analyzer (available from Texture Technologies Corporation) connected to a computer programmed with the texture analyzer software. The method parameters for the test, are outlined below in Table 1, the results of which are referred to herein as the TA Peak Force.

TABLE 1

| Parameter | Definition | Specification | |
|---|---|---|---|
| Mode | The type of test being run | Force/Comp. | |
| Option | The type of test sequence being run | Hold Until | |
| | | Time | |
| Distance | The set height at which the probe | 50.0 | mm |
| | resides prior to testing and after the test | | |
| | is complete | | |
| Pretest | How fast the probe travels at the start of | 4.00 | mm/sec |
| Speed | the run until the trigger point is reached | | |
| Trigger | The point at which a set minimal force | 1.0 | g |
| Point | is measured by the load cell as the | | |
| | probe contacts the sample surface. | | |
| | When the trigger force is reached, the | | |
| | test is started. | | |
| Speed | How fast the probe travels after the | 0.50 | mm/sec |
| | trigger point through compression to the | | |
| | designated force | | |
| Time | The amount of time that the probe | 140.00 | sec |
| | resides on the sample surface after the | | |
| | force has been achieved | | |
| Post - Speed | The speed at which the probe returns | 8.00 | mm/sec |
| | to the starting point where the test is | | |
| | finished | | |

| | | |
|---|---|---|
| Probe | The type of ball probe used for testing | TA-8 ¼″ |

During sample preparation, liquid adhesive is direct coated onto a siliconized liner and dried. The dried adhesive is then layered onto a 50 micron thick polyester film until a thickness of 280±25 micrometers has been achieved. During the test, the TA-8 probe (¼″ ball probe) moves into contact with the surface of the adhesive at a downward rate of 4.00 mm/second until a trigger force of 1 gram is registered on the load cell. The adhesive layer is then compressed to 50% of its total thickness at a rate of 0.50 millimeters/second which triggers data collection for the next 140 seconds. Throughout the entire test, the instrument continuously measures the change in force over time. Data on force versus time is generated into a graph on the computer. The initial peak force is the force required to compress the adhesive layer to 50% of its total thickness. The percent loss is related to the change in force over time (i.e., the change in initial force to the final force over the 140 second period of data collection).

Urethane elastomer pressure sensitive adhesives in accordance with exemplary embodiments are formulated so that a TA Peak Force between the range of about 40 to 180 grams and a percent loss in the range about 40 to 95% is obtained. In some embodiments, the TA Peak Force is between the range of 50 to 100 grams. Typically the percent loss is in the range of 45 to 95%, more typically in the range of 45% to 75% and preferably in the range of 50% to 65%. At TA Peak Force values greater than 180 grams, the average pain experienced during removal increases beyond 2.0 on the Wong-Baker scale. At a peak force less than 40 grams, the polyurethane adhesive was so compliant that no peel force to the skin could be measured (<0.1 N/cm), and the resultant wear performance suffered. When the percent loss of the adhesives went above 95% loss, the adhesive would leave a residue on the skin; at values less than 40% loss, the adhesive is so cohesive it did not satisfactorily wet out the skin surface and the material would fall off during wear testing.

The strength of an adhesive bond can be quantified as a mathematical product of two parameters: 1) the adhesive's ability to make the bond and 2) the adhesive's resistance to break the bond. Highest bond strengths are obtained when the adhesive easily flows to conform to the substrate to form a bond and then resists de-bonding by storing the de-bonding energy, achieving each of these two parameters. Conversely, a restricted adhesive flow prevents optimum contact with the substrate with respect to the first parameter and if the adhesive is also highly dissipative, it will not be in a position to resist debonding with respect to the second parameter and this combination will result in poor bond strength. The remaining combination of the two parameters will result in intermediate bond strength (i.e., an easily conforming adhesive that is also highly dissipative or an adhesive with restricted flow but with high energy storage ability).

Without wishing to be bound by theory, the inventors believe that exemplary embodiments of the invention having the compliance characteristics set forth herein with respect to TA Peak Force and percent loss correspond to a balance of the parameters that provides the excellent combination of properties exhibited.

In any event, once formulated, adhesives in accordance with exemplary embodiments may be coated as a continuous film onto a carrier substrate using any one of the several methods known to those skilled in the art for casting an adhesive film. The adhesive may be applied to a thickness of about 20 microns to about 100 microns, typically in the range of about 25 microns to about 80 microns, more typically in the range of about 30 to about 50 microns. The adhesive may be applied as a continuous or a discontinuous coating. The coating is subsequently dried/cured by passing it through a hot air oven. Alternatively, the coating may be exposed to UV radiation to affect curing. The adhesive film and/or the dressing or other device to which it is applied may be subjected to gamma radiation or other sterilization treatment without an adverse effect on the adhesive or its properties.

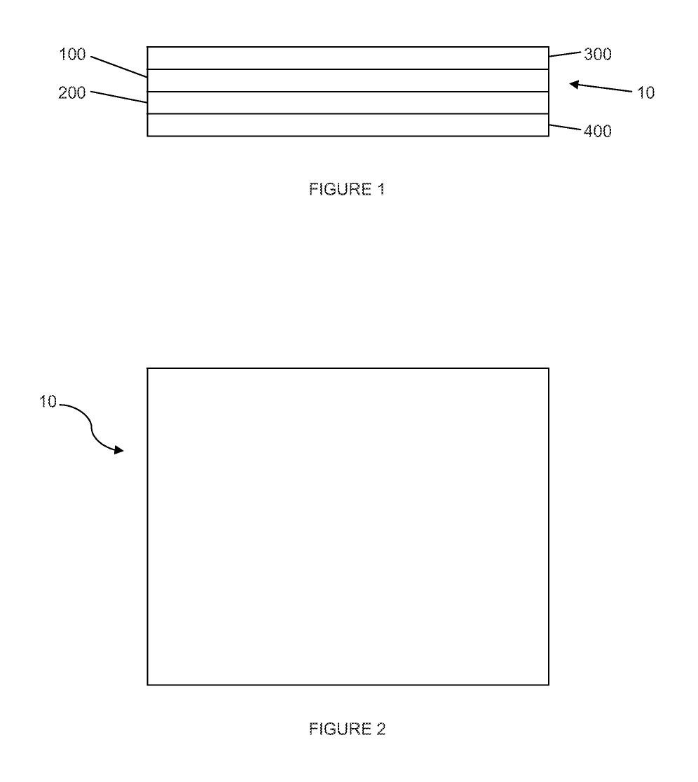

As shown in FIGS. 1 and 2, adhesive 100 may be applied directly to a substrate 200 to form a medical dressing 10 or other device, or be formed as a single or double sided tape for use in the later manufacture of a medical dressing or for direct use as an affixing tape to secure the dressing. A siliconized or other release liner 300 can be applied overlying the adhesive layer 100 to protect it prior to use.

In some embodiments, the substrate 200 to which the adhesive is applied is a thin polymeric film, such as a polyurethane film. These films are flexible and can follow the surface contours of the skin. In still other embodiments, a support layer 400 of paper, plastic or other suitable material may be used to improve handleability of the polymeric film prior to its initial application, which can then be peeled away.

### EXAMPLES

The invention is further described by way of the following examples, which are presented by way of illustration, not of limitation.

### Example 1

43.06 grams (71.2% by weight) of an aliphatic urethane acrylate oligomer (BR-3641AA from Bomar Specialties), 10.77 grams (17.8% by weight) of 1,2,3-triacetoxypropane, 6.46 (10.7% by weight) grams of cis-9-octadecen-1-ol, and 0.22 grams Irgacure 1700 (from Ciba) were mixed until homogeneous. The resulting mass was then placed on 50 micron polyester film and 50 micron siliconized polyester liner. The mass of adhesive between the films was then pulled between 2 coating bars separated with a 350 micron feeler gauge. The resulting adhesive mass was then cured with the films in place using a 300 W/in UV lamp equipped with a H bulb using a conveyor speed of 20 fpm. The sample was passed under the UV lamp two times per side, 4 times total.

The adhesive was characterized by compliance according to the TA Peak Force methodology described herein, yielding a peak force of 54 grams, and a percent loss of 67%.

### Example 2

64.33 grams (50% by dry weight) of the polyurethane pressure sensitive adhesive Cyabine SP-205 were blended with 0.53 grams of Cyabine T-501B, 32.16 grams (50% by dry weight) of capric/caprylic triglycerides, 0.141 grams Irganox 1010 and 1.13 grams of Irganox 3052FF to yield a clear solution. The resulting solution was coated on a 30 micron polyurethane film by passing between coating bars separated by a 300 micron feeler gauge. The resulting coating was dried in an oven at 120° C. for 5 minutes. The same coating was also coated on 50 micron siliconized polyester liner using a 300 micron feeler gauge. After drying at 120° C. for 5 minutes, the transfer film was laminated and layered on 50 micron polyester film to an adhesive thickness of 250 microns for compliance testing.

The adhesive of Example 2 was also characterized by compliance according to the TA Peak Force methodology described herein, yielding a peak force of 86 grams, and a percent loss of 58%.

An experiment was carried out on ten voluntary test subjects to measure the pain experienced upon removal of certain tapes to which an adhesive was applied. Several types of comparative commercial medical adhesives were used along with a tape using the adhesive formulated in accordance with Example 2. One commercial sample was a Tegaderm® negative pressure would therapy drape (an approximately 20 micron acrylic PSA on 25 micron polyurethane film commercially available from 3M) and another was a dressing made using an approximately 250 micron layer of a silicone gel adhesive (commercially available as Dow Corning Soft Skin 9850) on a 30 micron polyurethane film. The silicone gel adhesive, when coated at a thickness of 25-50 microns, has very low adhesion and tends to fall off. In order to increase the adhesion, commercial silicone adhesives are generally coated at greater thicknesses, and the silicone adhesive used as a comparative example in the experiments was applied to the manufacturer's recommended coating thickness of 250 microns.

The adhesive of Example 2 was coated to a thickness of approximately 50 microns and applied to a 30 micron polyurethane film.

Each of these samples was applied to each test subject's forearm and left in place for 24 hours. The samples were peeled back at an angle of 90° and the peel speed was 12 inches/minute in a tensile tester. The pain level was evaluated using the Wong-Baker pain scale. The amount of skin cells remaining on the adhesive after peeling from the forearm was also evaluated and graded according to the 0-5 scale reflected in Table 2 below.

TABLE 2

| Assessment Scale | Description |
|---|---|
| 0 | Few visible skin cells, <1% patch area |
| 1 | Some visible skin cells, <10% patch area |
| 2 | Moderate visible skin cells, <50% patch area |
| 3 | Nearly full coating of skin cells, |
| | still some tack to PSA |
| 4 | Full coating of skin cells, no tack to PSA |
| 5 | Full coating of skin cells and body hair with |
| | visible removal of skin from forearm of body |

The results of the experiment are reflected in Table 3:

TABLE 3

| | Tegaderm ® | Dow Corning | Example 2 |
|---|---|---|---|
| | negative pressure | Soft skin 9850 on | on 30 micron |
| | wound therapy | 30 micron | polyurethane |
| | drape | polyurethane film | film |
| Pain Ranking | 4.1 | 0 | 0.9 |
| Peel (N/cm) | 0.90 | 0.33* | 0.95 |
| Skin Cell | 3.8 | 0.1 | 0.1 |
| Assessment (0-5) | | | |
| Edge Lift (% | 0% | 50% | 0% |
| area) | | | |

*The silicone adhesive fell off in 50% of the test subjects within 24 hrs of application and these were not counted in the peel average.

The MVTR for each of the three adhesives was measured using the upright Payne cup method at 20% RH and 37° C. The results of the MVTR experiment are shown below in Table 4.

TABLE 4

| | Tegaderm ® | Dow Corning | |
|---|---|---|---|
| | negative pressure | Soft skin 9850 on | Example 2 |
| | wound therapy | 30 micron | on 30 micron |
| | drape | polyurethane film | polyurethane film |
| MVTR | 636 | 321 | 1280 |
| (g/m²-day) | | | |

The effect of gamma sterilization is determined by the change in peel adhesion value before and after treatment. It is desired that little or no change occur after treatment which signifies that the adhesive is resistant to the treatment. If the peel value changes then the performance of the adhesive in the final product cannot be predicted reliably. To compensate for this change in performance in conventional adhesives the formulators often error on the aggressive side and hope that the adhesive returns within a workable performance range after gamma sterilization. This leads to product variability and can result in a quality control issue, a manufacturing nightmare. Furthermore, the gamma treatment process itself cannot be controlled precisely. The gamma treatment dose for the medical industry varies from 25 to 40 kGy which makes controlling the adhesive performance even more troublesome. The effect of the gamma sterilization experiment on peels is shown below in Table 5, with the performance of the adhesive of Example 2 formulated in accordance with exemplary embodiments of the invention shows that the adhesive remains stable when subjected to gamma treatment.

TABLE 5

| | | Dow | Example 2 |
|---|---|---|---|
| | Medical grade | Corning Soft skin | on 30 micron |
| | Acrylic | 9850 on 30 micron | polyurethane |
| | adhesive | polyurethane film | film |
| Peel before gamma | 208.7 | 30.1 | 84.8 |
| treatment | | | |
| (g/cm) | | | |
| Peel after ~25 kGy | 60.3 | Could not be | 97.1 |
| gamma treatment | | measured | |
| (g/cm) | | | |

While the invention has been described with reference to particular embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims and all other patentable subject matter contained herein.

1. A medical pressure sensitive adhesive comprising a polyurethane elastomer and a plasticizer, tackifier, or both, wherein the adhesive has compliance properties corresponding to a TA Peak Force in the range of 40 grams to 180 grams and a percent loss in the range of 40% to 95%.

2. The adhesive of claim 1, wherein the polyurethane elastomer is comprised of a plurality of urethane linkages and is substantially free of isocyanate functionality.

3. The adhesive of claim 1, wherein the polyurethane elastomer has a weight average molecular weight of greater than 25,000 g/mol.

4. The adhesive of claim 1, wherein the polyurethane elastomer has a weight average molecular weight in the range of 50,000 g/mol to 150,000 g/mol.

5. The adhesive of claim 1, comprising about 10% to about 60% by weight plasticizer.

6. The adhesive of claim 1, wherein the polyurethane elastomer and plasticizer are biocompatible.

7. An article comprising a substrate and a medical pressure sensitive adhesive laminated to the substrate, the adhesive comprising a polyurethane elastomer and a plasticizer, tackifier, or both, wherein the adhesive has compliance properties corresponding to a TA Peak Force in the range of 40 grams to 180 grams and a percent loss in the range of 40% to 95%.

8. The article of claim 7, wherein the adhesive has a thickness in the range of about 20 to about 100 microns.

9. The article of claim 7, wherein the article has a stripping effect of a maximum of 20% when the adhesive is applied to human skin.

10. The article of claim 7, wherein the average pain rating upon removal of the adhesive from human skin is less than 2.5 on the Wong Baker pain scale.

11. The article of claim 7, wherein the adhesive exhibits a moisture vapor transmission rate value of greater than 1000 g/m²-day.

12. The article of claim 7, wherein the article is a wound dressing.

13. The article of claim 12, wherein the substrate is a polyurethane film.

14. The article of claim 7, wherein the article exhibits edge lift of less than 5% after 24 hours when applied to human skin.

15. The article of claim 7, wherein the adhesive has a peel strength of greater than 0.8 N/cm after 24 hours when applied to human skin.

16. A medical article comprising
a substrate;
a pressure sensitive adhesive having a thickness of about 25 to about 50 microns laminated to the substrate, the pressure sensitive adhesive comprising a polyurethane elastomer and a plasticizer, the adhesive having compliance properties corresponding to a TA Peak Force in the range of 40 grams to 180 grams and a percent loss in the range of 40% to 95%,
wherein the article has a stripping effect of a maximum of 20% when the adhesive is applied to human skin, the average pain rating upon removal of the adhesive from human skin is less than 2.5 on the Wong Baker pain scale, the adhesive exhibits a moisture vapor transmission rate value of greater than 1000 g/m²-day, the article exhibits edge lift of less than 5% after 24 hours when applied to human skin, and the adhesive has a peel strength of greater than 0.8 N/cm after 24 when applied to human skin.

17. A method of manufacturing a medical pressure sensitive adhesive comprising:
providing a polyurethane elastomer base pressure sensitive adhesive; and
modifying the adhesive with a plasticizer, tackifier, or both, to achieve compliance properties of the adhesive corresponding to a TA Peak Force in the range of 40 grams to 180 grams and a percent loss in the range of 40% to 95%.
